(19) United States
(12) Patent Application Publication
(10) Pub. No.: US 2004/0009465 A1
(43) Pub. Date: Jan. 15, 2004

(54) Shelf life indicator components for fresh cut fruits and vegetables responding to carbondioxide

(75) Inventor: Voraphat Luckanatinvong, Umphur Muang (TH)

Correspondence Address: Mr. Voraphat Luckanatinvong, Faculity of Science and Technology, Thammasat University, Klong Luang, Pathumthani, 12120, TH

(73) Assignee: Voraphat Luckanatinvong, Klong Luang (TH)

(21) Appl. No.: 10/615,951

(22) Filed: Jul. 10, 2003

(30) Foreign Application Priority Data
Jul. 12, 2002 (TH) 075156

(51) Int. Cl. C12Q 1/00; C12M 1/34

(52) U.S. Cl. 435/4; 435/287.1

(57) ABSTRACT

Shelf life indicator for fresh cut fruits and vegetables which reacts with carbondioxide inside the package. The absorbents are organic and inorganic that are not react with organic and inorganic indicators and carbondioxide. The permeability material that proper for carbondioxide to enter to react with the indicators and also prevents the indicators to contact with fresh cut fruits and vegetables inside the package.

### CROSS-REFERENCE TO RELATED APPLICATION

Not Applicable

### STATEMENT REGARDING FEDERALLY SPONSORED RESEARCH OR DEVELOPMENT

Not Applicable

### REFERENCE TO SEQUENCE LISTING

Not Applicable

### BACKGROUND OF THE INVENTION

Fresh fruits and vegetables which are cut and prepared ready for cook or eat fresh are increasingly demanded by the consumers. However there is problem of shelf life, it is not easy to determine. Under estimation of the shelf life causes the higher production cost and over estimation causes brand reputation. Sometime prepared fresh fruits and vegetables are found senescence before the expire date on the packages. The package of prepared fresh cut is usually wrapped with polyvinyl chloride (PVC) films. The carbondioxide and oxygen permeability of PVC film is not suitable for cut fruits and vegetables which are highly respiration.

Two types of modified atmosphere packaging (MAP) are active and passive modifications. Cut fruits and vegetables actually pack in passive modification. Respiration of perishable goods in passive modification consumes oxygen and generates carbondioxide inside the package. When the carbondioxide level increases and oxygen decreases, the inner package respiratory system will switch to anaerobic. Anaerobic respiratory causes the flavor change and reduces the quality therefore decrease the shelf life of the cut fruits and vegetables.

### DETAILED DESCRIPTION OF THE INVENTION

After anaerobic respiration occurred in the package of fresh cut, that causes of senescence of fresh cut. So it was lost quality and shelf life. The symptom was not predicted by labeled time on the package. We try to invent the indicator to detect shelf life of fresh cut which is responded with CO₂ concentrate in the package. So it causes to anaerobic respiration. The indicator is describing:

1. The indicator was made of Methyl red and Bromthymol blue at the concentration of 0.001 to 1.0 w/v. They changed from green to reddish orange color due to CO₂ concentration in the package.

2. The mixture used NaHCO₃ as indicator buffer at the concentrate of 0.001 to 1.0 molar. It controls equilibrium of acidity and alkalinity of solution of indicator that react with CO₂.

3. Phenolic compound and it's derivatives from plants or synthetic are Anthocyanin, Flavones and it's derivative, Betalien and it's derivative, Chlorophyll and it's derivative, Carotene, and Lycopene, at the concentrate of 0.001 to 10 w/v.

4. Distillation water dissolved the mixture.

After mixed and dissolved the ingredients, the green, yellow, blue or purple color was occurred due to type and concentration of the mixture.

| Example | |
|---|---|
| Mehtyl red | 0.01 g. |
| Bromthylmol blue | 0.005 g. |
| Anthocyanin | 0.01 g. |
| NaHCO₃ | 0.42 g. |
| Distillation water | enough to dissolve |

After prepared solution of indicator, that prepares the absorbent and sachet describes:

1. Indicator solution

2. Absorbent, it is made from plant fiber and celluloses which are purified and non purified material Or it is made from inorganic and organic material those do not react with ingredient of indicator.

3. Plastic, plant fiber and biodegradable plastic from starch base sachet, which are permeable for CO₂ transmission and it is not react with ingredient of indicator.

The indicator sachet is attached in the package of fresh cut that appear. When generated CO₂ in the fresh cut package was highly as it cause to anaerobic respiration. The indicator is changed green to red orange, red pink or red due to ingredient of indicator. It can detect the quality of fresh cut in the package as shelf life indicator. The customers or consumers can make decision to by the goods or not.

### BRIEF SUMMARY OF INVENTION

This invention provides a new shelf life indicator for fresh cut fruits and vegetables which reacts with carbondioxide inside the package. The absorbents are organic and inorganic that are not react with organic and inorganic indicators and carbondioxide. The permeability material that proper for carbondioxide to enter to react with the indicators and also prevents the indicators to contact with fresh cut fruits and vegetables inside the package.

We claim:

1. The components of shelf life indicator for fresh cut fruits and vegetable, which was responded to CO₂ concentration, was compose of Methyl red and/or Bromthylmol blue and/or NaHCO₃ buffer and/or Phenolic compound and/or Phenolic derivative and/or water and/or absorbent and/or Sachet.

2. The device of claim 1 wherein said the components were mixed together or used it only one.

3. The device of claim 1 and 2 wherein said methyl red was mixed in 0.001 to 1.0 percent of w/v.

4. The device of claim 1 to 3 wherein said Bromthylmal blue was mixed in 0.001 to 1.0 percent of w/v.

5. The device of claim 1 to 4 wherein said NaHCO₃ was mixed in 0.001 to 1.0 Malar

6. The device of claim 1 to 5 wherein said Phenolic compound and/or Phenolic derivative from plant and Synthetic were Anthocyanin, Plavonoid derivative, betalian and/or betalain derivative, Chlorophtyll, Lycopene and/or Carotenoid.

7. The device of claim 1 to 6 wherein said Phenolic and/or its derivative from plant and/or Synthetic was 0.001 to 10 percent or w/v.

8. The device of claim 1 to 7 wherein said water was dissolved.

9. The device of claim 1 to 8 wherein said the absorbent were plant fibers which were purified or non purified and/or inorganic and organic matter.

10. The device of claim 1 to 9 wherein said absorbent were inorganic and organic matter which did not reacted with the components of indicator.

11. The device of claim 1 to 10 wherein said the sachets were made from plastic and/or plant fiber and/or transparent films from starch base polymer that CO₂ permeated.

12. The device of claim 1 to 10 wherein said the indicator was changed color from green tone to orange or red orange tone. The green tone was signal for good quality of fresh cut but orange or red orange tone was signal for quality loss or shelf life loss.

13. The device of claim 1 to 10 wherein said the process was to mixed the indicator ingredient, then absorbent was immerse in the indicator solution and packed it to the sachet.

14. The device was from claim 13

15. The device of claim 14 wherein said the indicator appropriated for fresh cut fruits and vegetables which were packed in the packages.

16. The device was appropriate for indicate shelf life of fresh cut fruits and vegetables.
